(19) United States
(12) Patent Application Publication
(10) Pub. No.: US 2018/0201673 A1
(43) Pub. Date: Jul. 19, 2018

(54) TREATMENT OF PRURITUS

(71) Applicant: Eli Lilly and Company, Indianapolis, IN (US)

(72) Inventor: Matthew D Linnik, Solana Beach, CA (US)

(21) Appl. No.: 15/742,542

(22) PCT Filed: Jul. 7, 2016
(86) PCT No.: PCT/US2016/041277

§ 371 Date: Jan. 8, 2018

Related U.S. Application Data
(60) Provisional application No. 62/193,335, filed on Jul. 16, 2015.

(51) Int. Cl. C07K 16/24; A61P 17/04

(52) U.S. Cl. CPC C07K 16/244; A61K 2039/505; C07K 2317/76; A61P 17/04

(57) ABSTRACT

The present invention provides a method using IL-17 antagonistic antibodies for treating pruritus.

The present invention is in the field of medicine. More particularly, the present invention relates to the treatment of pruritus. More specifically, the present invention relates to the administration of IL-17 antagonistic antibodies to treat pruritus in a patient in need thereof. More specifically, the present invention relates to the administration of IL-17 antagonistic antibodies to treat pruritus associated with dermatological, systemic, neuropathic, or psychogenic disorders.

Pruritus is an uncomfortable skin sensation that provokes a desire to scratch. It is a common and distressing symptom in a variety of conditions and diseases. Pruritus is broadly categorized into four major causes; dermatological causes, systemic causes, neuropathic causes and psychogenic causes.

In practice, treatments of itch are divided into topical therapies, systemic therapies, phototherapy and behavioral therapy. Topical therapies include, but are not limited to, emollients and soaps, anesthetics such as capsaicin, pramoxine, lidocaine and prilocaine, coolants, glucocorticoids, calcincurin inhibitors and other agents. Systemic therapies include, but are not limited to, sedating antihistamines, anticonvulsants, antidepressants and opioid antagonists. Phototherapy includes the use of UV irradiation. Behavioral therapy includes counselling with a psychotherapist. Although the understanding of the pathogenesis of pruritus has improved significantly in recent years, pruritus therapy is still mostly based on conventional dermatologic therapies, some specific topical agents which act directly on nerves in the pathogenesis of pruritus, and substances acting not primarily on neurons. This underscores the need for the development of new substances that intervene specifically in the pathogenesis of pruritus.

Recently, attention has been given to antibody-based anti-cytokine therapy. For example, in spontaneous atopic dermatitis model mice NC/Nga, the blood concentration of IL-18 increases with advancement of the pathological conditions. However, continuous administrations of anti-IL-18 antibody tended to lead to an exacerbation of dermatitis and scratching behavior (Higa et al., British Journal of Dermatology 2003; 149: 39-45). More recently, antibodies against IL-31 and NR10 (the receptor for IL-31) have been proposed to treat pruritus (see U.S. Pat. Nos. 8,431,127 and 8,846,039, respectively). In a phase 2 study, use of an anti-IL-17 monoclonal antibody known as ixekizumab improved the clinical symptoms of psoriasis. Patients with chronic moderate-to-severe plaque psoriasis treated with ixekizumab had significant improvement in clinical measures (Psoriasis Area-and-Severity Index (PASI) and Static Physician's Global Assessment (sPGA)) during the 12-week treatment period that were rapid and sustained through 20 weeks with continued treatment (Leonardi et al., the New England Journal of Medicine 2012, 366: 1190-9). In that same study, patient reported secondary endpoints revealed a reduction in itch following treatment for their psoriasis. Patients were not required to have psoriasis-associated pruritus to enter the study, and patients were not being treated specifically for pruritus. Thus, there remains a need for novel therapies specifically for treating patients suffering from pruritus.

This invention provides a method for treating pruritus comprising administering to a patient with pruritus a therapeutically effective amount of an IL-17 antagonistic antibody. In another embodiment, the present invention provides a method of treating pruritus associated with dermatological, systemic, neuropathic, or psychogenic disorders comprising administering to a patient with pruritus a therapeutically effective amount of an IL-17 antagonistic antibody. In another embodiment, the IL-17 antagonistic antibody of the above methods comprises a light chain and a heavy chain, wherein the light chain comprises a light chain variable region (LCVR) and the heavy chain comprises a heavy chain variable region (HCVR), wherein the LCVR comprises LCDR1, LCDR2, and LCDR3, and the HCVR comprises HCDR1, HCDR2, and HCDR3, wherein:
a. LCDR1 comprises the amino acid sequence of SEQ ID NO: 1, LCDR2 comprises the amino acid sequence of SEQ ID NO: 2, and LCDR3 comprises the amino acid sequence of SEQ ID NO: 3, and HCDR1 comprises the amino acid sequence of SEQ ID NO: 4, HCDR2 comprises the amino acid sequence of SEQ ID NO: 5, and HCDR3 comprises the amino acid sequence of SEQ ID NO: 6; or
b. LCDR1 comprises the amino acid sequence of SEQ ID NO: 11, LCDR2 comprises the amino acid sequence of SEQ ID NO: 12, and LCDR3 comprises the amino acid sequence of SEQ ID NO: 13, and HCDR1 comprises the amino acid sequence of SEQ ID NO: 14, HCDR2 comprises the amino acid sequence of SEQ ID NO: 15, and HCDR3 comprises the amino acid sequence of SEQ ID NO: 16; or
c. LCDR1 comprises the amino acid sequence of SEQ ID NO: 21, LCDR2 comprises the amino acid sequence of SEQ ID NO: 22, and LCDR3 comprises the amino acid sequence of SEQ ID NO: 23, and HCDR1 comprises the amino acid sequence of SEQ ID NO: 24, HCDR2 comprises the amino acid sequence of SEQ ID NO: 25, and HCDR3 comprises the amino acid sequence of SEQ ID NO: 26.

In some embodiments, the IL-17 antagonistic antibody of the above methods comprises the LCVR amino acid sequence of SEQ ID NO: 7 and the HCVR amino acid sequence of SEQ ID NO: 8; or the LCVR amino acid sequence of SEQ ID NO: 17 and the HCVR amino acid sequence of SEQ ID NO: 18; or the LCVR amino acid sequence of SEQ ID NO: 27 and the HCVR amino acid sequence of SEQ ID NO: 28.

In some embodiments, the IL-17 antagonistic antibody of the above methods comprises the light chain amino acid sequence of SEQ ID NO: 9 and the heavy chain amino acid sequence of SEQ ID NO: 10, or the light chain amino acid sequence of SEQ ID NO: 19 and the heavy chain amino acid sequence of SEQ ID NO: 20; or the light chain amino acid sequence of SEQ ID NO: 29 and the heavy chain amino acid sequence of SEQ ID NO: 30.

Furthermore, this invention provides an IL-17 antagonistic antibody for use in the treatment of pruritus. In another embodiment, the present invention provides an IL-17 antagonistic antibody for use in the treatment of pruritus associated with dermatological, systemic, neuropathic, or psychogenic disorders. In another embodiment, the IL-17 antagonistic antibody of the above uses comprises a light chain and a heavy chain, wherein the light chain comprises a light chain variable region (LCVR) and the heavy chain comprises a heavy chain variable region (HCVR), wherein the LCVR comprises LCDR1, LCDR2, and LCDR3, and the HCVR comprises HCDR1, HCDR2, and HCDR3, wherein:
a. LCDR1 comprises the amino acid sequence of SEQ ID NO: 1, LCDR2 comprises the amino acid sequence of SEQ ID NO: 2, and LCDR3 comprises the amino acid sequence of SEQ ID NO: 3, and HCDR1 comprises the amino acid sequence of SEQ ID NO: 4, HCDR2 comprises the amino acid sequence of SEQ ID NO: 5, and HCDR3 comprises the amino acid sequence of SEQ ID NO: 6; or
b. LCDR1 comprises the amino acid sequence of SEQ ID NO: 11, LCDR2 comprises the amino acid sequence of SEQ ID NO: 12, and LCDR3 comprises the amino acid sequence of SEQ ID NO: 13, and HCDR1 comprises the amino acid sequence of SEQ ID NO: 14, HCDR2 comprises the amino acid sequence of SEQ ID NO: 15, and HCDR3 comprises the amino acid sequence of SEQ ID NO: 16; or
c. LCDR1 comprises the amino acid sequence of SEQ ID NO: 21, LCDR2 comprises the amino acid sequence of SEQ ID NO: 22, and LCDR3 comprises the amino acid sequence of SEQ ID NO: 23, and HCDR1 comprises the amino acid sequence of SEQ ID NO: 24, HCDR2 comprises the amino acid sequence of SEQ ID NO: 25, and HCDR3 comprises the amino acid sequence of SEQ ID NO: 26.

In some embodiments, the IL-17 antagonistic antibody of the above uses comprises the LCVR amino acid sequence of SEQ ID NO: 7 and the HCVR amino acid sequence of SEQ ID NO: 8; or the LCVR amino acid sequence of SEQ ID NO: 17 and the HCVR amino acid sequence of SEQ ID NO: 18; or the LCVR amino acid sequence of SEQ ID NO: 27 and the HCVR amino acid sequence of SEQ ID NO: 28.

In some embodiments, the IL-17 antagonistic antibody of the above uses comprises the light chain amino acid sequence of SEQ ID NO: 9 and the heavy chain amino acid sequence of SEQ ID NO: 10, or the light chain amino acid sequence of SEQ ID NO: 19 and the heavy chain amino acid sequence of SEQ ID NO: 20; or the light chain amino acid sequence of SEQ ID NO: 29 and the heavy chain amino acid sequence of SEQ ID NO: 30.

In another embodiment, the present invention also provides the use of an IL-17 antagonistic antibody for the manufacture of a medicament for the treatment of pruritus. In another embodiment, the present invention provides the use of an IL-17 antagonistic antibody for the manufacture of a medicament for the treatment of pruritus associated with dermatological, systemic, neuropathic, or psychogenic disorders.

An IL-17 antagonistic antibody refers to an antibody that binds IL-17 or the receptor thereof, and inhibits the activities of IL-17. IL-17 refers to IL-17A, IL-17B, IL-17C, IL-17D, IL-17E, or IL-17F and includes homodimers, heterodimers, or multimers of all IL-17 forms. IL-17 receptors refers to IL-17 receptor A, IL-17 receptor C. IL-17 receptor B, IL-17 receptor E, homomeric complexes, and heteromeric complexes thereof. Preferably, the IL-17 antagonistic antibody binds IL-17A homodimers, IL-17F homodimers, IL-17A/F heterodimers, or heteromeric complex of IL-17 receptor A and IL-17 receptor C. The embodiments of the IL-17 antagonistic antibodies are disclosed, for example, ixekizumab (see U.S. Pat. Nos. 7,838,638 and 8,110,191), secukinumab (see U.S. Pat. No. 7,807,155), and brodalumab (see U.S. Pat. No. 7,767,206).

Embodiments of antibodies that may be used to treat pruritus by blocking the interaction of IL-17 with its receptor include the following (the amino acid sequences of CDRs, variable regions as well as light chains and heavy chains are listed, respectively):

Ixekizumab
(LCDR1)
>SEQ ID NO: 1
RSSRSLVHSRGNTYLH
(LCDR2)
>SEQ ID NO: 2
KVSNRFI
(LCDR3)
>SEQ ID NO: 3
SQSTHLPFT
(HCDR1)
>SEQ ID NO: 4
GYSFTDYHIH
(HCDR2)
>SEQ ID NO: 5
VINPMYGTTDYNQRFKG
(HCDR3)
>SEQ ID NO: 6
YDYFTGTGVY
(LCVR)
>SEQ ID NO: 7
DIVMTQTPLSLSVTPGQPASISCRSSRSLVHSRGNTYLHWYLQKPGQSPQ
LLIYKVSNRF
IGVPDRFSGSGSGTDFTLKISRVEAEDVGVYYCSQSTHLPFTFGQGTKLE
IK
(HCVR)
>SEQ ID NO: 8
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTINTVSS
(light chain)
>SEQ ID NO: 9
DIVMTQTPLSLSVTPGQPASISCRSSRSLVHSRGNTYLHWYLQKPGQSPQ
LLIYKVSNRF
IGVPDRFSGSGSGTDFTLKISRVEAEDVGVYYCSQSTHLPFTFGQGTKLE
IKRTVAAPSV
FIFPFSDEQLKSGTASVVCLLNNFYPREAKVQWKVDNALQSGNSQESVFE
QDSKDSTYSLSSTLTLSKADYEKHKVYACEVTHQGLSSPVTKSFNRGEC
(heavy chain)
>SEQ ID NO: 10
QVQLVQSGAEVKKPGSSVKVSCKASGYSFTDYHIHWVRQAPGQGLEWMGV
INPMYGTTDYNQRFKGRVTITADESTSTAYMELSSLRSEDTAVYYCARYD
YFTGTGVYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTI
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLG
Secukinumab
(LCDR1)
>SEQ ID NO: 11
RASQSVSSSYLA
(LCDR2)
>SEQ ID NO: 12
GASSRAT
(LCDR3)
>SEQ ID NO: 13
QQYGSSPCT
(HCDR1)
>SEQ ID NO: 14
GFTFSNYWMN
(HCDR2)
>SEQ ID NO: 15
AINQDGSEKYYVGSVKG
(HCDR3)
>SEQ ID NO: 16
DYYDILTDYY1HYWYFDL
(LCVR)
>SEQ ID NO: 17
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPCTFG
QGTRLEIK
(HCVR)
>SEQ ID NO: 18
EVQLVESGGGLVQPGGSLRLSCAASGFTFSNYWMNWVRQAPGKGLEWVAA
INQDGSEKYYWISVKGRFFISRDNAKNSLYLQMNSLRVEDTAVYYCVRDY
YDILTDYYIHYWYFDLWGRGTLVTVSS
(light chain)
>SEQ ID NO: 19
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPCTFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
(heavy chain)
>SEQ ID NO: 20
EVQLVESGGGLVQPGGSLRLSCAASGFTFSNYWMNWVRQAPGKGLEWVAA
INQDGSEKYYVGSVKGRFTISRDNAKNSLYLQMNSLRVEDTAVYYCVRDY
YDILTDYYIHYWYFDLWGRGTLVTVSSASTKGPSVFPLAPSSKSTSGGTA
ALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTIPPSREEMTKNQVSLTCLVKGFYPSDIAVEWESNGQPENN
YKTTPPVLDS
DGSFFLYSKLTVDKSRWQQGNVFSCSVMHEALHNHYTQKSLSLSPGK
Brodalumab
(LCDR1)
>SEQ ID NO: 21
RASQSVSSNLA
(LCDR2)
>SEQ ID NO: 22
DASTRAT
(LCDR3)
>SEQ ID NO: 23
QQYDNWPLT
(HCDR1)
>SEQ ID NO: 24
GYTFTRYGIS
(HCDR2)
>SEQ ID NO: 25
WISTYSGNTNYAQKLQG
(HCDR3)
>SEQ ID NO: 26
RQLYFDY
(LCVR)
>SEQ ID NO: 27
EIVMTQSPATLSVSPGERATLSCRASQSVSSNLAWFQQKPGQAPRPLIYD
ASTRATGVPARFSGSGSGTDFTLTISSLQSEDFAVYYCQQYDNWPLTFGG
GTKVEIK
(HCVR)
>SEQ ID NO: 28
QVQLVQSGAEVKKPGASVKVSCKASGYTFTRYGISWVRQAPGQGLEWMGW
ISTYSGNTNYAQKLQGRVTMTTDTSTSTAYMELRSLRSDDTAVYYCARRQ
LYFDYWGQGTLVTVSS
(light chain)
>SEQ ID NO: 29
EIVMTQSPATLSVSPGERATLSCRASQSVSSNLAWFQQKPGQAPRPLIYD
ASTRATGVPARFSGSGSGTDFTLTISSLQSEDFAVYYCQQYDNWPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEK HKVYACEVTHQ
GLSSPVTKSFNRGEC
(heavy chain)
>SEQ ID NO: 30
QVQLVQSGAEVKKPGASVKVSCKASGYTFTRYGISWVRQAPGQGLEWMGW
ISTYSGNTNYAQKLQGRVTMTTDTSTSTAYMELRSLRSDDTAVYYCARRQ
LYFDYWGQGTLVTVSSASTKGPSVFPLAPCSRSTSESTAALGCLVKDYFP
EPVTVSWNSGALTSGVHTFPAVLQSSGLYSLSSVVTVPSSNFGTQTYTCN
VDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTFRVVSV
LTVVHQDWLNGKEYKCKVSNKGLPAPIEKTISKTKGQPREPQVYTLPPSR
EEMTKNQVSLTCLVKGFYPSDIAVEWESNGQPENNYKTTPPMLDSDGSFF
LYSKLTVDKSRWQQGNVFSCSVMHEALHNHYTQKSLSLSPGK

In some embodiments of the present invention, pruritus from dermatological causes includes atopic eczema, xerosis, scabies, contact dermatitis, insect bite, burn-induced pruritus, lichen planus, aquagenic pruritus, atopic dermatitis, impetigo, tinea, idiopathic pruritus, lichen simplex chronicus, allergic dermatoses, pruritic dermatoses, vascular dermatoses, sebaceous gland disorders, papulosquamous dermatoses, bacterial dermatoses, viral dermatoses, mycolic skin infections, granulomatous dermatoses, parasitic skin dermatoses, pediculosis corporis and pubis, exfoliative dermatitis, bullous dermatoses, pigmented dermatoses, photosensitive dermatoses, xerosis, wound, sun burn, cold sores, acne, insect bite, prurigo nodularis, primary skin cancer, metastatic skin cancer, nervous dermatitis, contact dermatitis, seborrheic dermatitis, autosensitization dermatitis, caterpillar dermatitis, asteatosis, insect sting, photosensitive dermatosis, and notalgia paresthetica.

In some embodiments of the present invention, pruritus from systemic causes includes chronic kidney disease, chronic kidney failure, liver disease, cholestasis, Hodgkin's lymphoma, polycythemia vera, HIV infections, herpes, rheumatoid arthritis, urticaria, systemic lupus erythematosus, progressive systemic sclerosis, Sjogren's syndrome, dermatomyositis, mixed connective tissue disease, multiple sclerosis, dermatoses caused by collagen diseases, dermatoses due to internal diseases, thyrotoxicosis, diabetes, renal insufficiency, uremia, hemodialysis, peritoneal dialysis, iron deficiency anemia, cholestasis chemotherapy, paraneoplastic syndrome, malignancy, hyperthyroidism, primary biliary cirrhosis, primary sclerosing cholangitis, obstructive choledocholithiasis, carcinoma of the bile duct, cholestasis, chronic hepatitis C viral infection and other forms of viral hepatitis, and pregnancy.

In some embodiments of the present invention, pruritus from neuropathic causes includes brachioradial pruritus, nostalgia paresthetica, and postherpetic itch.

In some embodiments of the present invention, pruritus from psychogenic causes includes obsessive-compulsive disorder, delusions of parasitosis, and substance abuse.

A therapeutically effective amount generally refers to an amount of an IL-17 antagonistic antibody that is effective, upon single or multiple dose administration to a patient at treating pruritus.

The IL-17 antagonistic antibodies, such as ixekizumab, secukinumab, or brodalumab, may be used as a pharmaceutical composition with a pharmaceutically acceptable carrier, excipient or diluent.

A pharmaceutical composition comprising ixekizumab is at a concentration in the range of about 80 mg/mL to about 150 mg/mL. A preferred ixekizumab concentration is in the range of about 68 mg/mL to about 92 mg/mL. A more preferred ixekizumab concentration is about 80 mg/mL. Another more preferred ixekizumab concentration is about 120 mg/mL. Another more preferred ixekizumab concentration is about 150 mg/mL.

A pharmaceutical composition comprising ixekizumab may also comprise a citrate buffer at a concentration in the range of about 15 mM to about 25 mM. A preferred concentration of citrate buffer is about 15 mM. Another preferred concentration of citrate buffer is about 20 mM. Another preferred concentration of citrate buffer is about 25 mM. Another preferred concentration of citrate buffer is about 30 mM. Citrate buffer can be made with citric acid, trisodium citrate dihydrate, and citric acid monohydrate; or citric acid monohydrate, sodium phosphate dibasic, and citric acid. Also, citrate buffer can be made comprising sodium citrate monobasic, citric acid trisodium salt, or sodium citrate tribasic hydrate. Preferably, citrate buffer is made with sodium citrate dihydrate and citric acid.

A pharmaceutical composition comprising ixekizumab may also comprise NaCl at a concentration in the range of about 200 mM to about 300 mM. A preferred NaCl concentration range about 175 mM to 225 mM. A preferred NaCl concentration is about 200 mM. Another preferred NaCl concentration is about 250 mM. Another preferred NaCl concentration is about 300 mM.

A pharmaceutical composition comprising ixekizumab may also comprise polysorbate-80 or polysorbate-20 at a concentration in the range of about 0.01% to about 0.04%. A preferred polysorbate-80 or polysorbate-20 concentration in the range of about 0.02% to about 0.04%. A preferred polysorbate-80 or polysorbate-20 concentration is about 0.03%. Another preferred polysorbate-80 or polysorbate-20 concentration is about 0.01%. Another preferred polysorbate-80 or polysorbate-20 concentration is about 1.2%. Another preferred polysorbate-80 or polysorbate-20 concentration is about 0.04%.

A pharmaceutical composition comprising ixekizumab may also have a pH range of about 5.4 to about 6.0. A preferred pH range is about 5.7. Another preferred pH range is about 5.4. Another preferred pH range is about 5.7. Another preferred pH range is about 6.0.

### Study Design

A double-blind, multicenter, randomized, dose-ranging study may be designed to evaluate the safety and efficacy of multiple subcutaneous doses of ixekizumab in patients with pruritus.

### Study Patients

Eligibility criteria may be a patient of age of 18 years or older and a clinical diagnosis of pruritus. Patients may be randomly assigned to receive subcutaneous injections of placebo or 10 mg, 25 mg, 75 mg, or 150 mg of ixekizumab at 0, 2, 4, 8, 12, and 16 weeks. As shown in Table 1, a dose dependent reduction in pruritus is observed upon treatment with ixekizumab that was evident beginning with the low dose 10 mg group. Mean pruritus scores appeared similar for doses of 25 mg, 75 mg and 150 mg at Week 8 and Week 16.

TABLE 1
Effect of Ixekizumab on Pruritus in Patients
with Moderate to Severe Plaque Psoriasis

| | Base- | Week | | Week | |
|---|---|---|---|---|---|
| Pruritus score | line | 8 | Change | 16 | Change |
| Placebo (n = 26) | 57.69 | 50.72 | −6.68 | 62.68 | 5.28 |
| 10 mg ixekizumab (n = 28) | 57.5 | 32.79 | −24.71 | 35.61 | −21.89 |
| 25 mg ixekizumab (n = 30) | 54.53 | 19.86 | −35.86 | 21.03 | −34.69 |
| 75 mg ixekizumab (n = 29) | 52.9 | 15.97 | −36.93 | 13.55 | −39.34 |
| 150 mg ixekizumab (n = 28) | 66.11 | 19.29 | −46.82 | 18.79 | −47.32 |

1. A method of treating pruritus comprising administering to a patient with pruritus a therapeutically effective amount of an IL-17 antagonistic antibody, wherein said antibody comprises a light chain and a heavy chain, wherein the light chain comprises a light chain variable region (LCVR) and the heavy chain comprises a heavy chain variable region (HCVR), wherein the LCVR comprises LCDR1, LCDR2, and LCDR3, and the HCVR comprises HCDR1, HCDR2, and HCDR3, wherein:
a. LCDR1 comprises the amino acid sequence of SEQ ID NO: 1, LCDR2 comprises the amino acid sequence of SEQ ID NO: 2, and LCDR3 comprises the amino acid sequence of SEQ ID NO: 3, and HCDR1 comprises the amino acid sequence of SEQ ID NO: 4, HCDR2 comprises the amino acid sequence of SEQ ID NO: 5, and HCDR3 comprises the amino acid sequence of SEQ ID NO: 6; or
b. LCDR1 comprises the amino acid sequence of SEQ ID NO: 11, LCDR2 comprises the amino acid sequence of SEQ ID NO: 12, and LCDR3 comprises the amino acid sequence of SEQ ID NO: 13, and HCDR1 comprises the amino acid sequence of SEQ ID NO: 14, HCDR2 comprises the amino acid sequence of SEQ ID NO: 15, and HCDR3 comprises the amino acid sequence of SEQ ID NO: 16; or
c. LCDR1 comprises the amino acid sequence of SEQ ID NO: 21, LCDR2 comprises the amino acid sequence of SEQ ID NO: 22, and LCDR3 comprises the amino acid sequence of SEQ ID NO: 23, and HCDR1 comprises the amino acid sequence of SEQ ID NO: 24, HCDR2 comprises the amino acid sequence of SEQ ID NO: 25, and HCDR3 comprises the amino acid sequence of SEQ ID NO: 26.

2. The method of claim 1, wherein the pruritus is associated with dermatological, systemic, neuropathic, or psychogenic disorders.

3. The method of claim 1, wherein the IL-17 antagonistic antibody comprises the LCVR amino acid sequence of SEQ ID NO: 7 and the HCVR amino acid sequence of SEQ ID NO: 8; or the LCVR amino acid sequence of SEQ ID NO: 17 and the HCVR amino acid sequence of SEQ ID NO: 18; or the LCVR amino acid sequence of SEQ ID NO: 27 and the HCVR amino acid sequence of SEQ ID NO: 28.

4. The method of claim 3, wherein the IL-17 antagonistic antibody comprises the light chain amino acid sequence of SEQ ID NO: 9 and the heavy chain amino acid sequence of SEQ ID NO: 10, or the light chain amino acid sequence of SEQ ID NO: 19 and the heavy chain amino acid sequence of SEQ ID NO: 20; or the light chain amino acid sequence of SEQ ID NO: 29 and the heavy chain amino acid sequence of SEQ ID NO: 30.

5. (canceled)

6. The antibody of claim 4, wherein the pruritus is associated with dermatological, systemic, neuropathic, or psychogenic disorders.

7. (canceled)

8. (canceled)

9. A method of treating pruritus comprising administering to a patient with pruritus a therapeutically effective amount of a pharmaceutical composition, wherein said pharmaceutical composition comprises a pharmaceutically acceptable carrier, excipient or diluent and an IL17 antagonistic antibody having a light chain and a heavy chain, wherein the light chain comprises a light chain variable region (LCVR) and the heavy chain comprises a heavy chain variable region (HCVR), wherein the LCVR comprises LCDR1, LCDR2, and LCDR3, and the HCVR comprises HCDR1, HCDR2, and HCDR3, wherein:
i. LCDR1 comprises the amino acid sequence of SEQ ID NO: 1, LCDR2 comprises the amino acid sequence of SEQ ID NO: 2, and LCDR3 comprises the amino acid sequence of SEQ ID NO: 3, and HCDR1 comprises the amino acid sequence of SEQ ID NO: 4, HCDR2 comprises the amino acid sequence of SEQ ID NO: 5, and HCDR3 comprises the amino acid sequence of SEQ ID NO: 6; or
ii. LCDR1 comprises the amino acid sequence of SEQ ID NO: 11, LCDR2 comprises the amino acid sequence of SEQ ID NO: 12, and LCDR3 comprises the amino acid sequence of SEQ ID NO: 13, and HCDR1 comprises the amino acid sequence of SEQ ID NO: 14, HCDR2 comprises the amino acid sequence of SEQ ID NO: 15, and HCDR3 comprises the amino acid sequence of SEQ ID NO: 16; or
iii. LCDR1 comprises the amino acid sequence of SEQ ID NO: 21, LCDR2 comprises the amino acid sequence of SEQ ID NO: 22, and LCDR3 comprises the amino acid sequence of SEQ ID NO: 23, and HCDR1 comprises the amino acid sequence of SEQ ID NO: 24, HCDR2 comprises the amino acid sequence of SEQ ID NO: 25, and HCDR3 comprises the amino acid sequence of SEQ ID NO: 26.

10. The method of claim 9, wherein the IL17 antagonistic antibody of the pharmaceutical composition is at a concentration in a range of about 80 mg/mL to about 150 mg/mL.

11. The method of claim 10 wherein the IL17 antagonistic antibody of the pharmaceutical composition is at a concentration of about 80 mg/mL.

12. The method of claim 10, wherein the pharmaceutical composition comprises citrate buffer at a concentration in a range of about 15 mM to about 25 mM.

13. The method of claim 12, wherein the citrate buffer is at a concentration of about 20 mM.

14. The method of claim 13, wherein the citrate buffer is derived from one of (a) citric acid, trisodium citrate dihydrate and citric acid monohydrate; (b) citric acid monohydrate, sodium phosphate dibasic and citric acid; (c) sodium citrate monobasic; (d) citric acid trisodium salt; (e) sodium citrate tribasic hydrate or (f) sodium citrate dihydrate and citric acid.

15. The method of claim 10, wherein the pharmaceutical composition comprises sodium chloride at a concentration in a range of about 200 mM to about 300 mM.

16. The method of claim 15, wherein the sodium chloride is at a concentration of about 200 mM.

17. The method of claim 10, wherein the pharmaceutical composition comprises one of polysorbate-80 or polysorbate-20 at a concentration in a range of about 0.01% to about 0.04% (w/v).

18. The method of claim 17, wherein the polysorbate-80 is at a concentration of about 0.03% (w/v).

19. The method of claim 10, wherein the pharmaceutical composition has a pH in a range of about 5.4 to about 6.0.

20. The method of claim 19, wherein the pH is about 5.7.

21. A method of treating pruritus comprising administering to a patient with pruritus a therapeutically effective amount of a pharmaceutical composition, wherein said pharmaceutical composition comprises:
(a) citrate buffer at a concentration of about 20 mM;
(b) sodium chloride at a concentration of about 200 mM;
(c) polysorbate-80 at a concentration in a range of about 0.02% (w/v) to about 0.03% (w/v);
(d) a pH at about 5.7; and
(e) an IL17 antagonistic antibody having a light chain and a heavy chain, wherein the light chain comprises a light chain variable region (LCVR) and the heavy chain comprises a heavy chain variable region (HCVR), wherein the LCVR comprises LCDR1, LCDR2, and LCDR3, and the HCVR comprises HCDR1, HCDR2, and HCDR3, wherein:
i. LCDR1 comprises the amino acid sequence of SEQ ID NO: 1, LCDR2 comprises the amino acid sequence of SEQ ID NO: 2, and LCDR3 comprises the amino acid sequence of SEQ ID NO: 3, and HCDR1 comprises the amino acid sequence of SEQ ID NO: 4, HCDR2 comprises the amino acid sequence of SEQ ID NO: 5, and HCDR3 comprises the amino acid sequence of SEQ ID NO: 6; or
ii. LCDR1 comprises the amino acid sequence of SEQ ID NO: 11, LCDR2 comprises the amino acid sequence of SEQ ID NO: 12, and LCDR3 comprises the amino acid sequence of SEQ ID NO: 13, and HCDR1 comprises the amino acid sequence of SEQ ID NO: 14, HCDR2 comprises the amino acid sequence of SEQ ID NO: 15, and HCDR3 comprises the amino acid sequence of SEQ ID NO: 16; or
iii. LCDR1 comprises the amino acid sequence of SEQ ID NO: 21, LCDR2 comprises the amino acid sequence of SEQ ID NO: 22, and LCDR3 comprises the amino acid sequence of SEQ ID NO: 23, and HCDR1 comprises the amino acid sequence of SEQ ID NO: 24, HCDR2 comprises the amino acid sequence of SEQ ID NO: 25, and HCDR3 comprises the amino acid sequence of SEQ ID NO: 26,
wherein the IL17 antagonistic antibody is at a concentration of about 80 mg/mL.

22. The method of claim 21, wherein the IL17 antagonistic antibody comprises the LCVR amino acid sequence of SEQ ID NO: 7 and the HCVR amino acid sequence of SEQ ID NO: 8; or the LCVR amino acid sequence of SEQ ID NO: 17 and the HCVR amino acid sequence of SEQ ID NO: 18; or the LCVR amino acid sequence of SEQ ID NO: 27 and the HCVR amino acid sequence of SEQ ID NO: 28.

23. The method of claim 21, wherein the IL-17 antagonistic antibody comprises the light chain amino acid sequence of SEQ ID NO: 9 and the heavy chain amino acid sequence of SEQ ID NO: 10, or the light chain amino acid sequence of SEQ ID NO: 19 and the heavy chain amino acid sequence of SEQ ID NO: 20; or the light chain amino acid sequence of SEQ ID NO: 29 and the heavy chain amino acid sequence of SEQ ID NO: 30.


SEQUENCE LISTING (30 sequences)

SEQ ID NO: 1 - protein, 16 aa, Artificial Sequence
  synthetic construct
RSSRSLVHSRGNTYLH

SEQ ID NO: 2 - protein, 7 aa, Artificial Sequence
  synthetic construct
KVSNRFI

SEQ ID NO: 3 - protein, 9 aa, Artificial Sequence
  synthetic construct
SQSTHLPFT

SEQ ID NO: 4 - protein, 10 aa, Artificial Sequence
  synthetic construct
GYSFTDYHIH

SEQ ID NO: 5 - protein, 17 aa, Artificial Sequence
  synthetic construct
VINPMYGTTDYNQRFKG

SEQ ID NO: 6 - protein, 10 aa, Artificial Sequence
  synthetic construct
YDYFTGTGVY

SEQ ID NO: 7 - protein, 112 aa, Artificial Sequence
  synthetic construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIK

SEQ ID NO: 8 - protein, 119 aa, Artificial Sequence
  synthetic construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSS

SEQ ID NO: 9 - protein, 219 aa, Artificial Sequence
  synthetic construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 445 aa, Artificial Sequence
  synthetic construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 12 aa, Artificial Sequence
  synthetic construct
RASQSVSSSYLA

SEQ ID NO: 12 - protein, 7 aa, Artificial Sequence
  synthetic construct
GASSRAT

SEQ ID NO: 13 - protein, 9 aa, Artificial Sequence
  synthetic construct
QQYGSSPCT

SEQ ID NO: 14 - protein, 10 aa, Artificial Sequence
  synthetic construct
GFTFSNYWMN

SEQ ID NO: 15 - protein, 17 aa, Artificial Sequence
  synthetic construct
AINQDGSEKYYVGSVKG

SEQ ID NO: 16 - protein, 18 aa, Artificial Sequence
  synthetic construct
DYYDILTDYYIHYWYFDL

SEQ ID NO: 17 - protein, 108 aa, Artificial Sequence [sequence omitted]

SEQ ID NO: 18 - protein, 127 aa, Artificial Sequence [sequence omitted]

SEQ ID NO: 19 - protein, 215 aa, Artificial Sequence
  synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 457 aa, Artificial Sequence
  synthetic construct
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 11 aa, Artificial Sequence
  synthetic construct
RASQSVSSNLA

SEQ ID NO: 22 - protein, 7 aa, Artificial Sequence
  synthetic construct
DASTRAT

SEQ ID NO: 23 - protein, 9 aa, Artificial Sequence
  synthetic construct
QQYDNWPLT

SEQ ID NO: 24 - protein, 10 aa, Artificial Sequence
  synthetic construct
GYTFTRYGIS

SEQ ID NO: 25 - protein, 17 aa, Artificial Sequence
  synthetic construct
WISTYSGNTNYAQKLQG

SEQ ID NO: 26 - protein, 7 aa, Artificial Sequence
  synthetic construct
RQLYFDY

SEQ ID NO: 27 - protein, 107 aa, Artificial Sequence
  synthetic construct
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 28 - protein, 116 aa, Artificial Sequence
  synthetic construct
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 29 - protein, 214 aa, Artificial Sequence
  synthetic construct
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 442 aa, Artificial Sequence
  synthetic construct
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK